(19) United States
(12) Patent Application Publication
(10) Pub. No.: US 2012/0238638 A1
(43) Pub. Date: Sep. 20, 2012

(54) BIOLOGICAL BIOCIDE ADDITIVES FOR POLYMERS

(76) Inventor: Jorma Antero Virtanen, Las Vegas, NV (US)

(21) Appl. No.: 13/423,459

(22) Filed: Mar. 19, 2012

Related U.S. Application Data
(60) Provisional application No. 61/454,128, filed on Mar. 18, 2011.

(51) Int. Cl. A01N 31/08; C08G 59/14; C08C 19/00; C07C 65/01; C07C 69/88; C07G 1/00; A01P 15/00; C08F 8/00; C07C 39/04; B82Y 40/00

(52) U.S. Cl. 514/731; 525/132; 525/523; 568/717; 562/478; 560/68; 530/500; 977/896

(57) ABSTRACT

This invention pertains to a method of modifying biocides obtained from biological sources, namely polyphenols. The modified biocide may have protective groups to reduce or prevent the oxidation of these phenols. The protective groups may also control the rate of hydrolyzation with regards to biocide efficacy. This invention also includes a method wherein the modified biocide may be incorporated and stabilized into a substrate like plastic.

This application claims priority to the provisional application under U.S. Ser. No. 61/454,128, entitled BIOLOGICAL BIOCIDE ADDITIVES FOR POLYMER, filed Mar. 18, 2011, which is incorporated herein by reference.

### I. BACKGROUND OF THE INVENTION

A. Field of Invention

This invention pertains to a method of modifying biocides obtained from biological sources. This invention also pertains to a method wherein the modified biocide is incorporated into a substrate like plastic.

B. Description of the Related Art

Plants can be very resistant against microbes, namely bacteria, fungi, and algae. This fact is taken for granted, but actually it is remarkable, because plants lack most, if not all defense mechanisms that animals may have. Some plants noted for their resistance include redwood and cedar trees. Most notably, redwood (sequoia sempervirens) can live thousand years or more without been affected by microbes. Even dead redwood is resistant against fungus. One reason for this microbial resistance is a mixture of chemicals called polyphenols. In many cases, polyphenols can be a significant part of this defensive mixture.

Polyphenols are a diverse group of molecules containing multiple phenolic rings. Well-known examples may include gallic acid, tannic acid, tannins in general, sequerins, and lignin and its various modifications. Polyphenols are present in most plants and plant parts. They can be extracted from wood, straw, and bark. Polyphenols may also be obtained from compressing wood to get the wood oils and from wood pulping. Polyphenols are known for their biocidal properties. Polyphenols have also been used to fabricate biopolymers. Because polyphenols originate from natural sources, they may be desirable from an environmental and toxicity standpoint. For instance, the use of polyphenols may be considered for coatings in humid places, marine applications, toys, baby bottles, and/or plasticware in healthcare. However, a problem with these polyphenolic compounds has been its poor miscibility with many plastics, its fast oxidation, and its solubity into water. Only compounds that are at least partially exposed on the surface can have more effective biocidic activity, and polyphenols by themselves may not be as effective due to their solubility properties.

Although many biocide additives exist for polymers, alternatives and/or improvements to what is currently available may be desired. The growing resistance of microbes with respect to current biocides as well as environmental regulations may provide opportunities for polyphenols. However, the instability of biological polyphenols in polymers has prevented its wider use. The present invention provides methods for modifying and incorporating polyphenols into polymers. These polymers may be used in the production of plastics. The present invention may also provide a method of stabilization of the polyphenols within the substrate, which may help the polyphenols to remain within the substrate and protect it.

### II. SUMMARY OF THE INVENTION

According to one aspect of the present invention, a method is provided which can modify a polyphenol for use as a biocide in a substrate, comprising the steps of: selecting at least one protective group; incorporating the protective group onto the polyphenol; and forming a modified polyphenol.

One aspect of this invention is that the polyphenol is derived from a natural source.

Another aspect of this invention is that the natural source comprises at least one source of cultivated plants, uncultivated plants, woody plants, agricultural plants, and flowering plants.

Still another aspect of this invention is that the natural source comprises at least one source of annual plants, biennial plants, and perennial plants.

Yet another aspect of this invention is that the polyphenol is an extract and/or a powder from the natural source.

Still yet another aspect of this invention is that the polyphenol comprises gallic acid, tannic acid, tannins, sequerins, lignin, and/or modifications of lignin.

One aspect of this invention is that the substrate comprises at least one substrate of plastic, rubber, and a coating.

Another aspect of this invention is that the protective group comprises acyl, alkyl, silyl, phophoryl, phosphonyl, phosphoryl, sulfate, sulfite, arseno and/or sulfonyl groups.

Still another aspect of this invention is that the protective group is selectively chosen to control the rate of oxidation of the polyphenol.

Yet another aspect of this invention is that the protective group is selectively chosen to control the rate of hydrolysis of the protective group.

Still yet another aspect of this invention is that forming the modified polyphenol comprises esterification with aliphatic carboxylic acid, esterification with aromatic carboxylic acid, silylation, ether formation, phosphate formation, sulfate formation, and/or arsenate formation.

One aspect of this invention is that modification of the modified polyphenol is partially reversible when the polyphenol with the protective group is exposed to water, air, and/or light.

Another aspect of this invention is that modification provides increased solubility for the polyphenol.

Still another aspect of this invention is that modification provides easier incorporation into a substrate for the modified polyphenol.

Yet another aspect of this invention is that it further comprises the step of reacting the modified polyphenol with a polymer and/or a monomer.

Still yet another aspect of this invention is that it further comprises the step of chemically coupling the modified polyphenol to a substrate.

One aspect of this invention is that it further comprises the steps of selecting nanoparticles; and incorporating the modified polyphenol inside of the nanoparticles wherein the nanoparticles release the modified polyphenol.

Another aspect of this invention is that it provides a method of modifying phenol for use as a biocide, comprising the steps of: selecting at least one protective group; incorporating the protective group onto the phenol; forming a modified phenol; and polymerizing the modified phenol to form a modified polyphenol.

Still another aspect of this invention is that it provides a method of modifying a polyphenol for use as a biocide in a plastic, comprising the steps of: selecting at least one protective group comprising of acyl, alkyl, silyl, phophoryl, phosphonyl, phosphoryl, sulfate, sulfite, arseno and/or sulfonyl groups; incorporating at least one protective group onto a polyphenol comprised of at least one polyphenol of gallic acid, tannic acid, tannins, sequerins, lignin, and modifications of lignin; and forming a modified polyphenol, wherein the protective group and the modified polyphenol are selectively chosen to control the rate of oxidation of the polyphenol and to control the rate of hydrolysis of the protective group.

Yet another aspect of this invention is that it provides a composition for modifying a polyphenol for use as a biocide in a substrate comprising: a polyphenol; and a protective group comprising of acyl, alkyl, silyl, phophoryl, phosphonyl, phosphoryl, sulfate, sulfite, arseno and/or sulfonyl groups, wherein the polyphenol is modified by the protective group and forms a modified polyphenol. The polyphenol comprises gallic acid, tannic acid, tannins, sequerins, lignin, and/or modifications of lignin. The substrate comprises a plastic, rubber, and/or a coating. The formation of the modified polyphenol comprises at least one modification of esterification with aliphatic carboxylic acid, esterification with aromatic carboxylic acid, silylation, ether formation, phosphate formation, sulfate formation, and arsenate formation. The modification of the modified polyphenol is partially reversible when the polyphenol with a protective group is exposed to water, air, and/or light. The modification provides increased solubility for the polyphenol. The modification provides easier incorporation into a substrate for the modified polyphenol. Additionally, the modified polyphenol comprises about 0.01% to about 20% by weight of the substrate.

Still yet another aspect of this invention is that the modified polyphenol can be stabilized within a substrate.

Still other benefits and advantages of the invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, a preferred embodiment of which will be described in detail in this specification and illustrated in the accompanying drawings which form a part hereof and wherein.

FIG. 1 is an example showing the modification of a polyphenol with aliphatic alcohol groups in which a mild methylation and partial esterifications may occur to provide a modified polyphenol that may be stabilized in a substrate.

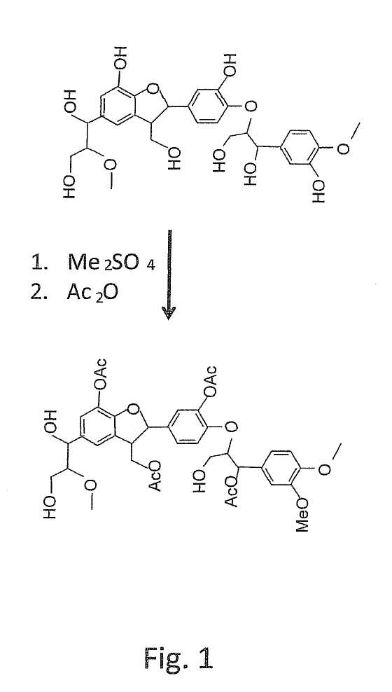

### IV. DETAILED DESCRIPTION OF THE INVENTION

Referring now to the drawings wherein the showings are for purposes of illustrating embodiments of the invention only and not for purposes of limiting the same, a method of modifying a polyphenol for use as a biocide in a substrate, comprising the steps of: (1) selecting at least one protective group, (2) incorporating the protective group onto the polyphenol, and (3) forming a modified polyphenol is described. Besides the modification of a polyphenol for use as a biocide in a substrate, a phenol may also be modified. A method for modifying a phenol may comprise the steps of: (1) selecting at least one protective group; (2) incorporating the protective group onto the phenol; (3) forming a modified phenol; and (4) polymerizing the phenol to form a modified polyphenol. In the invention described herein, the polyphenol may be used as a biocide. A biocide may be any compound that contains antipathogenic properties, prevents growth and/or the binding of bacteria, fungi, and/or algae, and/or acts as repellant against animals such as barnacles.

For the invention described herein, the modified polyphenols may be used as a biocide in plastic, rubber, and/or a coating. If a plastic is used, it may be thermoplastic or thermoset. Protected biocidic compounds described in this invention may be used as additives for substrates such as plastics, rubbers, and/or coatings. The modified polyphenols described herein may comprise about 0.01% by weight of the substrate to about 20% by weight of the substrate.

For the invention described herein, extracts or ground powders of polyphenols from a natural source may be used. However, this invention may not be limited to any specific compound or class of compounds, although polyphenols will be mostly used in the examples provided herein. Polyphenols are chemical compounds comprising a large number of phenol structural units. Polyphenols may be obtained from natural sources, including various plant sources. These plant sources may include woody plants, agricultural plants, cultivated plants, uncultivated plants, and/or flowering plants. These plant sources may also include annual plants, biennial plants, and perennial plants. Polyphenols from woody plants can be obtained from logs, wood chips, wood bark, wood powder, sawdust, pulp products, wood pellet products, sawmill products, salvaged wood products, logging waste, forest products, and/or wood products. Sources of woody plants can encompass both native and cultivated trees. Additionally, polyphenols from other plant sources may be obtained from aquatic plants, native and hybrid shrubs and bushes, and/or residential or commercial landscaping plants. Polyphenols from agricultural plants can include agricultural food and feed crops, and other agricultural products. Polyphenols from cultivated plants can include cultivated crop plants. Polyphenols may also be obtained from the by-products, residues, and/or waste products of plants. Peat and humus are plant residues that are stable against microbial and fungal degradation. Their fragments that can be obtained by chemical and/or thermal degradation can be used as starting materials in the present invention. Chemical degradation can be a combination of oxidative cleaving and reductive recovery of phenolic hydroxyl groups. Phenolic hydroxyls of the polyphenols may be protected by acyl, alkyl, silyl, phophoryl, phosphonyl, or sulphonyl groups. Other protective groups may include phosphoryl, sulfate, sulfite, and/or arseno groups.

Well known examples of polyphenols can be gallic acid, tannic acid, tannins in general, sequerins, and/or lignin and its various modifications. Tannins may be plant polyphenolic compound containing sufficient hydroxyls and other suitable groups such as carboxyls to form strong complexes with proteins and other macromolecules. Tannins can be found in leaf, bud, seed, root, and stem tissues of plants. Tannins may contain both gallic acid and/or tannic acid. For sequerins, they may be mostly obtained from redwood or other living trees. Lignin has a complex, polymeric structure whose exact structure is unknown. Lignin may be one of the most abundant source of aromatic chemicals. Lignin can be a structurally complex substance made up of p-hydroxybenzene, guaiacyl(4-alkyl-2-methoxyphenol), and syringyl(4-alkyl-2,5-dimethoxyphenol) units. The abundance of each of these units may change somewhat between individual species of plants. Lignin fragments can also be obtained as a side product from pulp cooking. Modifications of lignin may also be used.

Although polyphenols have been described almost exclusively herein, other bioactive molecules may behave analogously. For example, aliphatic alcohols may form more stable esters, but many of them may be hydrolyzed too quickly. Also, amines can form amides that are very stable, but dimethyl phosphoryl group may be hydrolyzed fast. In addition, photocleavable groups can be used to protect amino groups. These protecting groups are well known in the art (see P. G. M. Wuts and T. W. Greene, Greene's Protective Groups in Organic Synthesis, 4Th Ed., Wiley 2006). Also, phenols may also be modified with protective groups as described herein, then polymerized to form modified polyphenols.

For the method of modifying a phenol and/or polyphenol for use as a biocide in a substrate, the first step can be to obtain a protective group(s) for the phenols and/or polyphenols. The protective groups may be gradually hydrolyzed depending on the selection of at least one protective group. Protective groups can slow down and/or prevent oxidation for a polyphenol exposed to a humid or watery environment. The rate of hydrolysis can be adjusted by proper choice of the protecting group.

For modifying a phenol and/or polyphenol, the phenolic hydroxyls can be protected by acyl, alkyl, silyl, phophoryl, phosphonyl, and/or sulphonyl groups. Other protective groups may include phosphoryl, sulfate, sulfite, and/or arseno groups. These protective groups may slow down or prevent oxidation of phenols. In addition, most protective groups can increase the solubity of these additives into plastics and other substrates. In forming the modified polyphenols, at least one modification may include esterification with aliphatic carboxylic acid, esterification with aromatic carboxylic acid, silylation, ether formation, phosphate formation, sulfate formation, and arsenate formation. The modification process may provide both increased solubility for the polyphenol as well as an easier incorporation for the modified polyphenol into a substrate. With the modification of polyphenol, it may be partially reversible when the polyphenol with the protective group can be exposed to water, air, and/or light.

For the protective groups, arseno groups may be distinct from the other protective groups, because arsenate can be strongly toxic, and may be used only in some specific applications. These heteroatomic esters may be fully esterified. For example, phosphate can be esterified also with two alcohols, such as methanol, ethanol, 2-propanol, etc., including long chain alcohols. Hydrolysis of these fully esterified compounds can be much faster than that of partial esters. Hydrolysis of sulfates may be faster than that of other heteroesters. Also, solubility in most plastics can be improved. During hydrolysis, phenolic hydroxyl group may be exposed first in most cases, because it can be a better leaving group than the aliphatic alcohol group.

The rate of hydrolysis may vary between protective groups. The hydrolysis rate of various protective groups may have a range that covers several orders of magnitude, in practical terms hydrolysis can happen in seconds or last several years. In most cases, it may be advantageous to use a mixture of protecting groups in order to cover various time spans for hydrolysis rates. In one example, trimethyl silyl, formate, and/or trifluoroacetate, can last only minutes or hours, when exposed on the surface. In another example, acetate and/or methyl sulfate can last hours or days. In yet another example, benzoate and/or dimethyl phosphate can last weeks or months. With another example, methyl and/or benzyl can last several months, or more than year. For another example, phenyl ether may be stable several years. For all of these examples provided above, the time frames provided may be rough estimates, since the actual times can depend on the humidity, pH, salt concentration in water, duration of immersion, temperature, and/or exposure for sun light. Thus, the best combination of protective groups for a specific application may be found by experimentation. In one instance, the stability of the protective groups may not exceed stability of the surface itself. If one nanometer of a surface is worn out every day, and new molecules will be exposed every day, the stability of protective groups may be measured in minutes, or at most in hours. Otherwise, biocidic molecules can be removed before they are activated.

Although hydrolysis can play a role, sometimes hydrolysis may be too fast. The rate of hydrolysis can be related to the efficacy. The molecules of the protective groups that may be exposed on the surface can be hydrolyzed, resulting in a return to phenols. On the other hand, the embedded molecules can stay protected until the material may be worn so much that the protective groups may become exposed. Some water may penetrate the plastic, and embedded molecules may provide enough stability to resist hydrolysis.

Aliphatic carboxylic acids may be hydrolyzed faster than aromatic carboxylic acids. In general, small carboxylic acids can be hydrolyzed faster than larger carboxylic acids. For example, formates may be hydrolyzed faster than acetates, and acetates can be faster than propionates. Hexanoates can be hydrolyzed faster than benzoates.

There are several methods in which to modify the polyphenols and/or phenols. These may include but are not limited to alkylation, benzylation, arylation, and/or silylation.

Alkylation, for example, by dimethyl or diethyl sulfate can be easily performed in alkaline water solution. Benzylation by benzyl chloride may be another option. The ether bond can be stable against hydrolysis, but may not be as stable against oxidation. Triphenyl methyl group may be an exception, because it can be easily hydrolyzed. Alkylation and/or arylation may be used to increase solubility and/or long term stability, and only part of phenolic hydroxyl group may be protected using the ether bond. The small part of phenolic hydroxyl groups in each molecule may be protected by polyethylene glycol (PEG), because PEGylated surface can be a strongly hydrated, fuzzy surface that does not provide sticking points for pathogens, algae, and/or barnacles.

Silylation can be straightforward, in principle. Solubility of polyphenolic compounds may pose issues with certain chemicals. Ethyl acetate and other medium polarity solvents that do not have hydroxyl groups may be used. Hydrolysis of many silyl compounds can be fast. For example, dimethyl phenyl and methyl diphenyl groups may be used.

The chemistry of attaching the protecting groups may be well known in the art. A single phase reaction can be done. However, a low solubility may require a two-phase reaction. In attaching the protecting groups, a reagent such as ethyl acetate, diethyl ether, butyl acetate, dichloromethane, and/or cyclohexane may be in the organic phase while a polyphenol is in the water phase. Depending on the reaction, the water phase may be alkaline, so that phenols are in salt form. Trimethyl hexadecyl ammonium chloride may be used as a phase transfer agent. Other phase transfer agents, such as crown ethers, may equally well be used.

In addition, most protective groups may increase the solubility of these additives into plastics. Several types of protective groups may help to improve this solubility. By increasing the solubility, the modified polyphenol may be incorporated into the substrate easier.

After the protecting group is attached, the solution can be filtered. The solvent may or may not be removed before an optional mixing with a monomer and/or polymer. If the product of this invention may be mixed with monomer, some of it may be bound chemically with a monomer during polymerization.

When a modified polyphenol may be exposed on the surface, some of it or all of it may be removed, especially if the surface may be submerged in water. In order to avoid the removal of the modified polyphenol too quickly, some phenolic hydroxyl groups may be left unprotected, so that they can bind chemically with polymer. For example, an epoxy may react with a phenolic hydroxyl group, and a polyphenol can be bound with the epoxy matrix. In another example, if a polyphenol may be incorporated in acrylic monomer, such as methyl methacrylate, a protecting group like methacrylic acid may become a part of polymer chain during polymerization.

Some aromatic acids also may have antimicrobial activity. These can include benzoic acid and salicylic acid. The rate of hydrolysis of alkanoates may also depends on the substituent in 2-position. Steric hindrance can slow down the hydrolysis. Thus, the hydrolysis of 2-methyl propionate may be slower than that of butanoate. Electronegative substituents in 2-position can speed up the hydrolysis. For instance, the hydrolysis of trifluoroacetates can be very fast.

Besides providing a method for modifying a polyphenol, the invention herein also provides a method for modifying a phenol may comprise the steps of: (1) selecting at least one protective group; (2) incorporating the protective group onto the phenol; (3) forming a modified phenol; and (4) polymerizing the phenol to form a modified polyphenol. By starting with a phenol instead of a polyphenol, the modified polyphenol may still be produced by the process described herein.

After the protecting group is attached, the solution can be filtered. The solvent may or may not be removed before mixing with a monomer and/or polymer. If the product of this invention may be mixed with monomer, some of it may be bound chemically with a monomer during polymerization.

When a modified polyphenol may be exposed on the surface, some of it or all of it may be removed, especially if the surface may be submerged in water. In order to avoid the removal of the modified polyphenol too quickly, some phenolic hydroxyl groups may be left unprotected, so that they can bind chemically with polymer. For example, an epoxy may react with a phenolic hydroxyl group, and a polyphenol can be bound with the epoxy matrix. In another example, if a polyphenol may be incorporated in acrylic monomer, such as methyl methacrylate, a protecting group like methacrylic acid may become a part of polymer chain during polymerization.

Besides the methods claimed herein, a composition for modifying a polyphenol for use as a biocide is also claimed. This composition may comprise a polyphenol comprising of gallic acid, tannic acid, tannins, sequerins, lignin, and/or modifications of lignin; and a protective group comprising of acyl, alkyl, silyl, phophoryl, phosphonyl, phosphoryl, sulfate, sulfite, arseno and/or sulfonyl groups, wherein said polyphenol is modified by said protective group and forms a modified polyphenol. The modified polyphenol may comprise about 0.01% by weight to about 20% by weight of the substrate.

The substrate used for this composition may be comprised of plastic, rubber, and/or a coating. In forming the modified polyphenol, the reactions may include the modification of esterification with aliphatic carboxylic acid, esterification with aromatic carboxylic acid, silylation, ether formation, phosphate formation, sulfate formation, and/or arsenate formation. With the composition described, the modification of the modified polyphenol can partially reversible when the polyphenol with the protective group may be exposed to water, air, and/or light. This modification can provides both an increased solubility for the polyphenol and an easier incorporation into a substrate for the modified polyphenol.

Still another aspect of the present invention is to chemically couple a polyphenol with plastic, either thermoset resin of thermoplastic. With chemical coupling, the modified polyphenol may be chemically bound to the substrate such that it can help to keep it with the substrate. By coupling the polyphenol to it, leaching of the polyphenol from the substrate can be reduced. Also, chemical coupling may act to slow down oxidation.

The biocidic compounds described herein can be used in conjunction of many other additives such as nanoparticles. The nanoparticles may include carbon nanotubes (CNTs), inorganic nanotubes (INTs), porous plastics, alumina, silica, zeolite, and activated carbon. These nanoparticles may act as carriers and slow releasing agents for the biocides of present invention if the hollow parts of these nanoparticles are first filled with modified polyphenols. The modified polyphenols may be added inside the nanoparticles by using a vacuum to remove the air from pores.

The embodiments have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

Having thus described the invention, it is now claimed:

1. A method of modifying a polyphenol for use as a biocide in a substrate, comprising the steps of:
selecting at least one protective group;
incorporating said protective group onto said polyphenol; and
forming a modified polyphenol.

2. The method of claim 1, wherein said polyphenol is derived from a natural source.

3. The method of claim 2, wherein said natural source comprises at least one source of cultivated plants, uncultivated plants, woody plants, agricultural plants, and flowering plants.

4. The method of claim 2, wherein said natural source comprises at least one source of annual plants, biennial plants, and perennial plants.

5. The method of claim 2, wherein said polyphenol is an extract from said natural source.

6. The method of claim 2, wherein said polyphenol is a powder from said natural source.

7. The method of claim 1, wherein said polyphenol comprises at least one polyphenol of gallic acid, tannic acid, tannins, sequerins, lignin, and modifications of said lignin.

8. The method of claim 1, wherein said substrate comprises at least one substrate of plastic, rubber, and a coating.

9. The method of claim 1, wherein said protective group comprises at least one protective group of acyl, alkyl, silyl, phophoryl, phosphonyl, phosphoryl, sulfate, sulfite, arseno, and sulfonyl groups.

10. The method of claim 1, wherein said protective group is selectively chosen to control the rate of oxidation of said polyphenol.

11. The method of claim 1, wherein said protective group is selectively chosen to control the rate of hydrolysis of said protective group.

12. The method of claim 1, wherein said forming said modified polyphenol comprises at least one modification of esterification with aliphatic carboxylic acid, esterification with aromatic carboxylic acid, silylation, ether formation, phosphate formation, sulfate formation, and arsenate formation.

13. The method of claim 1, wherein said modification of said modified polyphenol is partially reversible when said polyphenol with said protective group is exposed to at least one factor of water, air, and light.

14. The method of claim 1, wherein said modification provides increased solubility for said polyphenol.

15. The method of claim 1, wherein said modification provides easier incorporation and stability with said substrate for said modified polyphenol.

16. The method of claim 1, further comprising the step of:
reacting said modified polyphenol with at least one chemical of a polymer and a monomer.

17. The method of claim 1, further comprising the step of:
chemically coupling said modified polyphenol to said substrate.

18. The method of claim 1, further comprising the steps of:
selecting nanoparticles; and
incorporating said modified polyphenol inside of said nanoparticles,
wherein said nanoparticles release said modified polyphenol.

19. A method of modifying a phenol for use as a biocide, comprising the steps of:
selecting at least one protective group;
incorporating said protective group onto said phenol;
forming a modified phenol; and
polymerizing said modified phenol to form a modified polyphenol.

20. A method of modifying a polyphenol for use as a biocide in a plastic, comprising the steps of:
selecting at least one protective group comprising at least one protective group of acyl, alkyl, silyl, phophoryl, phosphonyl, phosphoryl, sulfate, sulfite, arseno and sulfonyl groups;
incorporating said at least one protective group onto said polyphenol comprised of at least one polyphenol of gallic acid, tannic acid, tannins, sequerins, lignin, and modifications of lignin; and
forming a modified polyphenol,
wherein said protective group and said modified polyphenol are selectively chosen to control the rate of oxidation of said polyphenol and to control the rate of hydrolysis of said protective group.

21. A composition for modifying a polyphenol for use as a biocide in a substrate comprising:
a polyphenol comprising at least one polyphenol of gallic acid, tannic acid, tannins, sequerins, lignin, and modifications of said lignin; and
a protective group comprising at least one protective group of acyl, alkyl, silyl, phophoryl, phosphonyl, phosphoryl, sulfate, sulfite, arseno and sulfonyl groups,
wherein said polyphenol is modified by said protective group and forms a modified polyphenol.

22. The composition of claim 21, wherein said substrate comprises at least one substrate of plastic, rubber, and a coating.

23. The composition of claim 21, wherein said forming said modified polyphenol comprises at least one modification of esterification with aliphatic carboxylic acid, esterification with aromatic carboxylic acid, silylation, ether formation, phosphate formation, sulfate formation, and arsenate formation.

24. The composition of claim 21, wherein said modification of said modified polyphenol is partially reversible when said polyphenol with said protective group is exposed to at least one factor of water, air, and light.

25. The composition of claim 21, wherein said modification provides increased solubility for said polyphenol.

26. The composition of claim 21, wherein said modification provides easier incorporation into a substrate for said modified polyphenol.

27. The composition of claim 21, wherein said modified polyphenol comprises about 0.01% by weight to about 20% by weight of said substrate.
